(19) United States
(12) Patent Application Publication
(10) Pub. No.: US 2025/0206688 A1
(43) Pub. Date: Jun. 26, 2025

(54) METHOD FOR PRODUCING CHLOROTRIFLUOROETHYLENE AND TRIFLUOROETHYLENE

(71) Applicant: AGC INC., Tokyo (JP)

(72) Inventor: Kenya KASUGA, Chiyoda-ku (JP)

(73) Assignee: AGC INC., Tokyo (JP)

(21) Appl. No.: 19/081,095

(22) Filed: Mar. 17, 2025

Related U.S. Application Data
(63) Continuation of application No. PCT/JP2023/030282, filed on Aug. 23, 2023.

(30) Foreign Application Priority Data
Sep. 22, 2022 (JP) 2022-151981

(51) Int. Cl. C07C 17/23

(52) U.S. Cl. CPC C07C 17/23

(57) ABSTRACT

A production method for producing chlorotrifluoroethylene and trifluoroethylene, the method including reacting 1,1,2-trichloro-1,2,2-trifluoroethane with hydrogen in a gas phase in the presence of a diluent, in which a volume of the diluent to be supplied to a reactor at which the reaction is performed is from 0.4 times to 6 times a volume of the 1,1,2-trichloro-1,2,2-trifluoroethane to be supplied to the reactor.

### CROSS-REFERENCE TO RELATED APPLICATIONS

This application is a Continuation Application of International Application No. PCT/JP2023/030282, filed Aug. 23, 2023, the disclosure of which is incorporated herein by reference in its entirety. Further, this application claims priority from Japanese Patent Application No. 2022-151981, filed Sep. 22, 2022, the disclosure of which is incorporated herein by reference in its entirety.

### TECHNICAL FIELD

The present disclosure relates to a method for producing chlorotrifluoroethylene and trifluoroethylene.

### BACKGROUND ART

Patent Document 1 discloses a method of producing chlorotrifluoroethylene by reacting 1,1,2-trichloro-1,2,2-trifluoroethane with hydrogen in the presence of a catalyst. Hereinafter, 1,1,2-trichloro-1,2,2-trifluoroethane is also referred to as “CFC-113”, and chlorotrifluoroethylene is also referred to as “CTFE”.

In Patent Document 1, by using iron or an oxide of iron, nickel, copper, tin, or zinc as a catalyst, CTFE is obtained in a high yield, the activity of the catalyst is maintained for a long time, and the productivity is increased.

Trifluoroethylene can be produced, for example, by further reacting CTFE, which is obtained by the reaction between CFC-113 and hydrogen, with hydrogen. Trifluoroethylene may be directly obtained as a by-product in the reaction between CFC-113 and hydrogen for producing CTFE.
Patent Document 1: Japanese Patent Application Laid-Open (JP-A) No. H3-173840

### SUMMARY OF INVENTION

### Technical Problem

Trifluoroethylene has attracted attention as a refrigerant having a small global warming potential. Hereinafter, trifluoroethylene is also referred to as “HFO-1123”.

In order to efficiently obtain HFO-1123, in the reaction between CFC-113 and hydrogen, the conversion rate of CFC-113 as a raw material is required to be high, and the total selectivity of CTFE and HFO-1123 is required to be high.

An object of the disclosure is to provide a production method achieving both improvement in a conversion rate of CFC-113 and improvement in a total selectivity of CTFE and HFO-1123 in a reaction between CFC-113 and hydrogen.

### Solution to Problem

The disclosure includes the following aspects.

<1> A production method for producing chlorotrifluoroethylene and trifluoroethylene, the production method including reacting 1,1,2-trichloro-1,2,2-trifluoroethane with hydrogen in a gas phase in the presence of a diluent,
in which a volume of the diluent supplied to a reactor at which the reaction is performed is from 0.4 times to 6 times a volume of the 1,1,2-trichloro-1,2,2-trifluoroethane supplied to the reactor.

<2> The production method according to <1>, in which the diluent includes at least one selected from the group consisting of water vapor, nitrogen, argon, and helium.

<3> The production method according to <1>, in which the diluent includes water vapor.

<4> The production method according to any one of <1> to <3>, in which a catalyst is not provided inside the reactor.

<5> The production method according to any one of <1> to <4>, in which the volume of the diluent supplied to the reactor is from 3 times to 6 times the volume of the 1,1,2-trichloro-1,2,2-trifluoroethane supplied to the reactor.

<6> The production method according to any one of <1> to <5>, in which a volume of the hydrogen supplied to the reactor is from 0.3 times to 2 times the volume of the 1,1,2-trichloro-1,2,2-trifluoroethane supplied to the reactor.

<7> The production method according to any one of <1> to <6>, in which a temperature of the reaction is from 560° C. to 600° C.

<8> The production method according to any one of <1> to <7>, in which a time of the reaction is from 1 second to 12 seconds.

<9> The production method according to <3>, including removing at least a part of the water vapor from a gas composition obtained by the reaction, the gas composition including the chlorotrifluoroethylene, the trifluoroethylene, and the water vapor.

<10> The production method according to <9>, in which the removing includes liquefying at least a part of the water vapor at a temperature of 5° C. or lower.

<11> The production method according to <9> or <10>, in which a catalyst is not provided inside the reactor.

<12> The production method according to any one of <9> to <11>, in which the volume of the diluent supplied to the reactor is from 3 times to 6 times the volume of the 1,1,2-trichloro-1,2,2-trifluoroethane supplied to the reactor.

<13> The production method according to any one of <9> to <12>, in which a volume of the hydrogen supplied to the reactor is from 0.3 times to 2 times the volume of the 1,1,2-trichloro-1,2,2-trifluoroethane supplied to the reactor.

<14> The production method according to any one of <9> to <13>, in which a temperature of the reaction is from 560° C. to 600° C.

<15> The production method according to any one of <9> to <14>, in which a time of the reaction is from 1 second to 12 seconds.

<16> The production method according to any one of <1> to <15>, in which the 1,1,2-trichloro-1,2,2-trifluoroethane is obtained by fluorinating tetrachloroethylene with hydrogen fluoride in the presence of a catalyst.

### Advantageous Effects of Invention

According to the disclosure, there is provided a production method achieving both improvement in a conversion rate of CFC-113 and improvement in a total selectivity of CTFE and HFO-1123 in a reaction between CFC-113 and hydrogen.

### BRIEF DESCRIPTION OF DRAWINGS

FIG. 1 is a diagram illustrating an example of a reaction device used in a production method of the present disclosure.

### DESCRIPTION OF EMBODIMENTS

Hereinafter, embodiments of the disclosure will be described in detail. However, the disclosure is not limited to the following embodiments. In the following embodiments, constituent elements (including elemental steps and the like) are not necessarily indispensable unless otherwise stated. The same applies to numerical values and ranges thereof, and does not limit the disclosure.

In the present disclosure, the term “step” includes not only a step independent of other steps but also a step by which an action of the step is achieved, though the step cannot be clearly distinguished from other steps.

In the present disclosure, a numerical range that has been indicated by use of “to” includes the numerical values which are described before and after “to”, as a minimum value and a maximum value, respectively.

In the present disclosure, each component may contain plural corresponding substances. In a case in which plural substances corresponding to each component are present in the composition, the ratio of each component means a total ratio of the plurality of substances present in the composition unless otherwise specified.

In a case in which an embodiment is described with reference to the drawings in the present disclosure, the configuration of the embodiment is not limited to the configuration illustrated in the drawings. The sizes of members in each drawing are conceptual, and the relative relationship between the sizes of the members is not limited thereto.

### Production Method

A production method in an embodiment of the present disclosure is a method of producing chlorotrifluoroethylene (CTFE) and trifluoroethylene (HFO-1123) by reacting 1,1,2-trichloro-1,2,2-trifluoroethane (CFC-113) with hydrogen in a gas phase. In the present embodiment, the reaction is performed in the presence of a diluent, and a volume of the diluent supplied to a reactor at which the reaction is performed is from 0.4 times to 6 times a volume of CFC-113 supplied to the reactor.

Hereinafter, a ratio of the volume of the diluent supplied to the reactor to the volume of CFC-113 supplied to the reactor is also referred to as a “diluent ratio”.

Here, the reaction between CFC-113 and hydrogen in the production method of the embodiment is represented by the following formula.

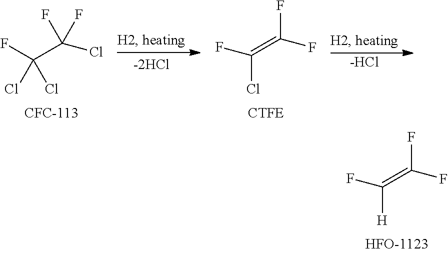

In the present embodiment, CFC-113 is used as a raw material, and CFC-113 and hydrogen are brought into contact with each other in a gas phase to produce CTFE and HFO-1123 together. The reaction is preferably performed under heating.

Specifically, in a case in which CFC-113 as a raw material is heated in the presence of hydrogen, CTFE is produced while producing two molecules of hydrogen chloride by a thermal hydrocracking reaction. It is considered that the generated CTFE further reacts with another molecule of hydrogen to generate HFO-1123. The “thermal hydrocracking reaction” refers to a reaction in which a hydrogen atom is introduced to a compound using a hydrogen gas as a reducing agent under a heating condition.

In the present embodiment, the reaction between CFC-113 and hydrogen is performed in the presence of a diluent, and the diluent ratio is set to from 0.4 times to 6 times, thereby achieving both improvement in a conversion rate of CFC-113 and improvement in a total selectivity of CTFE and HFO-1123.

Specifically, by setting the diluent ratio in the above range, dimerization due to an intermolecular reaction of CTFE is suppressed, and generation of CTFE and HFO-1123 is promoted. Therefore, it is considered that the total selectivity of CTFE and HFO-1123 is improved.

It is considered that by setting the diluent ratio in the above range, the concentration of hydrogen in the gas phase is higher than that in a case in which the diluent ratio is larger than the above range, and CFC-113 and hydrogen are easily brought into contact with each other, so that the conversion rate of CFC-113 is improved.

Hereinafter, the conversion rate of CFC-113 is also simply referred to as “conversion rate”, and the total selectivity of CTFE and HFO-1123 is also simply referred to as “total selectivity”.

### <Diluent>

The diluent is a component for adjusting the concentration of CFC-113 as a raw material to be low, and is a component other than CFC-113 as a raw material and hydrogen.

The diluent is not limited as long as it is a compound having low reactivity, and a high-boiling-point compound is preferable from the viewpoint of ease of separation from a product. The boiling point of the diluent is preferably higher than 47.5° C., which is the boiling point of CFC-113, at atmospheric pressure, more preferably 70° C. or higher, and still more preferably 90° C. or higher.

Specific examples of the diluent include water vapor, nitrogen, argon, helium, and carbon dioxide. As the diluent, any one of these may be used singly, or in combination of two or more thereof. The total content of water vapor, nitrogen, argon, helium, and carbon dioxide included in the diluent is preferably 90 vol % or more, more preferably 95 vol % or more, and still more preferably 99 vol % or more, with respect to the total amount of the diluent.

From the viewpoint of low reactivity, the diluent preferably includes at least one selected from the group consisting of water vapor, nitrogen, argon, and helium among these. The diluent more preferably includes at least one selected from the group consisting of water vapor and nitrogen.

Among them, the diluent more preferably includes water vapor. In a case in which the diluent includes water vapor, the total selectivity is further improved. The reason for this is not clear, but it is presumed that hydrogen chloride generated by reduction of CFC-113 and water vapor undergo an association action, and a side reaction between hydrogen chloride and CTFE is suppressed, so that byproducts are less likely to be generated. The diluent is preferably water vapor from the viewpoint of ease of separation. Since CTFE and HFO-1123, which are target substances, are components having a low boiling point, by using water vapor having a high boiling point as a diluent, separation between the target substance and the diluent becomes easy, and equipment cost of a separation step can be reduced.

The content of water vapor included in the diluent is preferably 50 vol % or more, more preferably 60 vol % or more, still more preferably 70 vol % or more, still more preferably 80 vol % or more, and still more preferably 90 vol % or more, with respect to the total amount of the diluent.

From the viewpoint of improving the total selectivity and the conversion rate, the diluent ratio, that is, the ratio of the volume of the diluent ratio to the volume of CFC-113 supplied to the reactor is from 0.4 times to 6 times, preferably from 1.5 times to 6 times, still more preferably from 3 times to 6 times, and further preferably from 4.5 times to 6 times.

In a case in which the production method of the present embodiment is a continuous method, the diluent ratio can be controlled by adjusting the flow rate of CFC-113 to the reactor and the flow rate of diluent to the reactor.

From the viewpoint of productivity of CTFE and HFO-1123, a ratio of the volume of the diluent to a total volume of CFC-113, hydrogen, and the diluent supplied to the reactor is preferably from 12 vol % to 82 vol %, and more preferably from 54 vol % to 71 vol %.

### <CFC-113>

From the viewpoint of productivity of CTFE and HFO-1123, a ratio of the volume of CFC-113 to the total volume of CFC-113, hydrogen, and the diluent supplied to the reactor is preferably from 11 vol % to 59 vol %, and more preferably from 12 vol % to 30 vol %.

CFC-113 is obtained, for example, by using tetrachloroethylene as a raw material and fluorinating tetrachloroethylene with hydrogen fluoride in the presence of a catalyst. That is, the production method of the present embodiment may include fluorinating tetrachloroethylene to obtain CFC-113 and reacting CFC-113 with hydrogen to obtain CTFE and HFO-1123.

Examples of the catalyst used for fluorination of tetrachloroethylene include antimony pentachloride.

Examples of the temperature at which tetrachloroethylene is fluorinated include from 45° C. to 200° C.

Examples of the pressure in the fluorination of tetrachloroethylene include from 100 kPa to 3500 kPa.

### <Hydrogen>

Hydrogen is a raw material that reacts with CFC-113 as a raw material in the reaction represented by the above formula.

A ratio of a volume of hydrogen to the volume of CFC-113 supplied to the reactor is preferably from 0.3 times to 2 times, more preferably from 1 time to 2 times, and still more preferably from 1.5 times to 2 times. Hereinafter, the ratio of the volume of hydrogen to the volume of CFC-113 supplied to the reactor is also referred to as a “hydrogen ratio”.

It is considered that in a case in which the hydrogen ratio is set in the above range, as compared with a case in which the hydrogen ratio is lower than the above range, CFC-113 and hydrogen are easily brought into contact with each other, so that the conversion rate is improved, dimerization due to an intermolecular reaction of CTFE is suppressed, and the total selectivity is improved. In a case in which the hydrogen ratio is set in the above range, as compared with a case in which the hydrogen ratio is higher than the above range, for example, side reactions of CFC-113 and CTFE and the like are less likely to proceed, an excessive reduction reaction of HFO-1123 is less likely to proceed, so that the total selectivity is improved. In a case in which the hydrogen ratio is in the above range, the volume of CFC-113 supplied to the reactor is larger than that in a case in which the hydrogen ratio is higher than the above range, so that the productivity of CTFE and HFO-1123 is improved. In a case in which the hydrogen ratio is in the above range, the amount of unreacted hydrogen is reduced as compared with a case in which the hydrogen ratio is higher than the above range, so that a load in the separation step described later can be reduced.

In a case in which the production method of the present embodiment is a continuous method, the hydrogen ratio can be controlled by adjusting the flow rate of CFC-113 to the reactor and the flow rate of hydrogen to the reactor.

From the viewpoint of improving the total selectivity, the ratio of the volume of hydrogen to the total volume of CFC-113, hydrogen, and the diluent supplied to the reactor is preferably 59 vol % or less, and more preferably 34 vol % or less. From the viewpoint of improving the conversion rate, the ratio of the volume of hydrogen to the total volume of CFC-113, hydrogen, and the diluent supplied to the reactor is preferably 4 vol % or more, and more preferably 18 vol % or more.

From the viewpoint of improving the conversion rate and the total selectivity, the ratio of the volume of hydrogen to the total volume of CFC-113, hydrogen, and the diluent supplied to the reactor is preferably from 4 vol % to 59 vol %, and more preferably from 18 vol % to 34 vol %.

### <Reaction Step>

In the production method of the present embodiment, the method of bringing CFC-113 and hydrogen into contact with each other is not particularly limited. The contact can be performed by supplying CFC-113 and hydrogen into a reaction field between CFC-113 and hydrogen, for example, a reactor. Hereinafter, the reaction field between CFC-113 and hydrogen will be described as a reactor. In the following description, unless otherwise specified, CFC-113 and hydrogen are in a gas phase state.

The production method of the present embodiment may be a continuous method or a batch method. In the continuous production method, for example, supplying CFC-113 as a raw material and hydrogen to the reactor, bringing CFC-113 and hydrogen into contact with each other in the reactor, and taking out CTFE and HFO-1123 from the reactor are all performed continuously.

In the continuous production method, the diluent ratio, the hydrogen ratio, and the ratio of volume of each component to the total volume of CFC-113, hydrogen, and the diluent supplied to the reactor are represented by the ratio of the volume flow rate per unit time.

In a case in which the production method of the present embodiment is a continuous method, the supply of CFC-113, hydrogen, and the diluent to the reactor may be performed separately, or may be performed by mixing in advance.

From the viewpoint of causing the reaction in a gas phase state and the viewpoint of preventing decomposition of CFC-113, the temperature of CFC-113 supplied to the reactor is preferably from 60° C. to 575° C., and more preferably from 60° C. to 200° C.

From the viewpoint of improving the reactivity between CFC-113 and hydrogen, it is preferable that hydrogen and the diluent are mixed in advance and heated in a preheater. The temperature of the mixture of hydrogen and the diluent supplied to the reactor is preferably from 100° C. to 600° C., more preferably from 300 to 600° C., and still more preferably from 575 to 600° C.

The shape and structure of the reactor that is used to bring CFC-113 and hydrogen into contact with each other are not particularly limited as long as it withstands the temperature and pressure described later, and examples thereof include a cylindrical vertical reactor. In a case in which the production method of the present embodiment is a continuous method, the cylindrical vertical reactor may be used as a reaction route.

Examples of the material of the reactor include iron and an alloy containing nickel as a main component. The reactor may include a heating means such as an electric heater.

In the present embodiment, a catalyst may be provided inside the reactor or may not be provided inside the reactor. In the present embodiment, it is preferable to not provide a catalyst inside the reactor. The phrase “a catalyst is not provided inside the reactor” means that the catalyst is not intentionally placed in a space inside the reactor, and includes a case in which a wall surface or the like of the reactor functions as a catalyst.

By not providing a catalyst inside the reactor, the cost is reduced as compared with the case of providing a catalyst, and it is not necessary to consider replacement of the catalyst due to catalyst deterioration, and the productivity of the CTFE and the HFO-1123 is improved.

In the present embodiment, since the reaction between CFC-113 and hydrogen is performed in the presence of a diluent and the diluent ratio is from 0.4 times to 6 times, the conversion rate and the total selectivity are improved without providing a catalyst inside the reactor.

In the present embodiment, a catalyst may be provided inside the reactor.

The temperature of the reaction between CFC-113 and hydrogen is preferably from 560° C. to 600° C., more preferably from 560 to 585° C., and still more preferably from 560 to 575° C. In a case in which the temperature of the reaction is in the above range, as compared with a case in which the temperature is lower than the above range, the conversion rate is improved. In a case in which the temperature of the reaction is in the above range, as compared with a case in which the temperature is higher than the above range, an excessive reaction such as carbonization of an organic substance is suppressed and the total selectivity is improved.

The temperature of the reaction is a temperature of a gas phase in the reactor, that is, a mixed gas in which CFC-113, hydrogen, and a diluent are mixed, and is a value measured by a thermocouple or the like.

The time of the reaction between CFC-113 and hydrogen is preferably from 1 second to 12 seconds, more preferably from 1 second to 8 seconds, and still more preferably from 1 second to 6 seconds. In a case in which the time of the reaction is in the above range, as compared with a case in which the time is shorter than the above range, the thermal hydrocracking reaction of CFC-113 sufficiently proceeds and the conversion rate is improved. In a case in which the time of the reaction is in the above range, as compared with a case in which the time is longer than the above range, CTFE and HFO-1123 can be produced with high productivity, and side reactions are suppressed, so that the total selectivity is improved.

The time of the reaction corresponds to the residence time of CFC-113 and hydrogen in the reactor. In a case in which the production method of the present embodiment is a continuous method, the time of the reaction is calculated from the total flow rate of CFC-113, hydrogen, and the diluent into the reactor and the volume of the reactor. The time of the reaction can be controlled by adjusting the supply amount (that is, flow rate) of CFC-113, hydrogen, and the diluent to the reactor.

The pressure in the reaction between CFC-113 and hydrogen is preferably normal pressure (that is, atmospheric pressure). The pressure in the reaction means the pressure in the reactor. In the present disclosure, unless otherwise specified, the pressure indicates a gauge pressure.

### <First Gas Composition>

In the thermal hydrocracking reaction in the reaction step of CFC-113 and hydrogen, a composition containing CTFE and HFO-1123 as target products is obtained as a first gas composition.

Examples of the compound other than CTFE and HFO-1123 contained in the first gas composition include, in addition to CFC-113, hydrogen, and the diluent as unreacted raw materials, by-products such as 1,1-difluoroethylene (HFO-1132a), 1,2-dichloro-1,2,3,3,4,4-hexafluorocyclobutane, 1,3-dichloro-1,2,2,3,4,4-hexafluorocyclobutane, 1,2-dichloro-1,1,2-trifluoroethane (HCFC-123a), 1,1-dichloro-1,2,2-trifluoroethane (HCFC-123b), 1,1-dichloro-2,2-difluoroethylene (CFO-1112), 1-chloro-2,2-difluoroethylene (HFO-1122), 1-chloro-2,2-difluoroethylene (HFO-1122a (E)), and 1-chloro-2,2-difluoroethylene (HFO-1122a (Z)).

### <Separation Step>

The production method of the present embodiment may include separating CTFE and HFO-1123, which are target substances, from components other than CTFE and HFO-1123 after reacting CFC-113 with hydrogen in a gas phase. That is, the production method of the present embodiment may include a separation step of separating components other than CTFE and HFO-1123 from the first gas composition.

### (Raw Material Separation Step)

The separation step may include, for example, separating a part or all of CFC-113 as a high-boiling component from the first gas composition to obtain a second gas composition having an increased content ratio of CTFE and HFO-1123. Hereinafter, the step of separating CFC-113 from the first gas composition to obtain a second gas composition is also referred to as “raw material separation step”.

A method of separating CFC-113 from the first gas composition is not particularly limited, and can be arbitrarily selected according to reaction conditions and reaction products. Examples thereof include liquefaction separation by heat removal to a temperature equal to or lower than the standard boiling point of CFC-113, liquefaction separation by heat removal to a temperature equal to or lower than the boiling point at the pressure under high pressure conditions, distillation such as extraction distillation, and an absorption method in which CFC-113 is dissolved in an absorbent. These methods may be performed in a single method or a combination of plural methods. In a case in which separation is performed by a single method, it may be performed in one step or in several steps.

In a case in which water vapor is used as the diluent, the separation step preferably includes removing at least a part of water vapor from the gas composition containing at least CTFE, HFO-1123, and water vapor. The removal of water vapor preferably includes liquefying at least a part of water vapor. By removing water vapor by liquefying at least a part of water vapor, the equipment for the subsequent process can be reduced. It is preferable that at least a part of water vapor is liquefied at a temperature of 5° C. or lower.

The removal of water vapor may be performed in conjunction with the separation of CFC-113 from the first gas composition. That is, in the raw material separation step, water vapor may be removed from the first gas composition together with CFC-113.

As a method of separating CFC-113 and water vapor from the first gas composition, it is preferable to perform liquefaction by heat removal under a slightly pressurized condition from the viewpoint of equipment. Heat removal may be performed directly or indirectly on CFC-113 and water vapor. CFC-113 and water recovered by liquefaction separation can be separated after two-phase separation into an organic phase and an aqueous phase.

In the case of liquefying water vapor in the raw material separation step, the conditions for liquefying are preferably in a range of a pressure of from 0 MPa to 0.55 MPa and a temperature of from −40° C. to 100° C. In particular, in a case in which both CFC-113 and water vapor are liquefied in the separation step, the liquefaction conditions are more preferably in a range of a pressure of from 0 MPa to 0.55 MPa and a temperature of from −40° C. to 48° C., and still more preferably in a range of a pressure of from 0 MPa to 0.55 MPa and a temperature of from −40° C. to 5° C.

In the raw material separation step, the second gas composition containing unreacted CFC-113 is obtained. The second gas composition may contain other compounds used in the reaction step, other components produced in the reaction step, and the like, in addition to CTFE and HFO-1123 as target substances and the unreacted CFC-113. In the raw material separation step, CFC-113 contained in the first gas composition and water vapor in the case of using water vapor as a diluent are selectively separated. Therefore, the content ratio of CTFE and HFO-1123 in the second gas composition is higher than the content ratio of CTFE and HFO-1123 in the first gas composition.

The second gas composition can be used as it is for various uses, and is preferably further purified.

Examples of the purification method include known methods such as distillation, adsorption, and washing with an acidic aqueous solution, a basic aqueous solution, or a neutral aqueous solution. Substances other than CTFE and HFO-1123 contained in the second gas composition can be removed by known means, and concentrations other than CTFE and HFO-1123 contained in the second gas composition can be adjusted. Preferably, the purification method is a method of distilling under normal pressure, under increased pressure, or under reduced pressure. By performing distillation under such a pressure, high-purity CTFE and HFO-1123 are obtained. The unreacted CFC-113 separated from the second gas composition can be recycled as a part of the raw material for the reaction step.

Examples of the method of separating unreacted hydrogen from the second gas composition include distillation and adsorption separation using an adsorbent.

In a case in which nitrogen is used as a diluent, examples of the method of separating nitrogen from the second gas composition include distillation and adsorption separation using an adsorbent.

In a case in which a rare gas such as argon or helium is used as a diluent, examples of the method of separating the rare gas from the second gas composition include distillation and adsorption separation using an adsorbent.

The water vapor separated in the raw material separation step can be recovered. The recovered water vapor can be reused again as a diluent in the reaction step.

A part of water vapor used as a diluent may be used as a water source in other steps other than the reaction step, or water used in various applications in other steps other than the reaction step may be used as a part of the diluent. For example, water vapor used as a diluent may be used as water which is a solvent of a basic aqueous solution used in a hydrogen chloride separation step described later, or water which is a solvent of a basic aqueous solution used for washing the second gas composition may be used as water vapor as a diluent in the reaction step.

### (Hydrogen Chloride Separation Step)

The separation step may include separating hydrogen chloride contained in the first gas composition. Hereinafter, the step of separating hydrogen chloride contained in the first gas composition is also referred to as a “hydrogen chloride separation step”. The hydrogen chloride separation step may be performed between the reaction step and the raw material separation step, may be performed simultaneously with the raw material separation step, or may be performed after the raw material separation step.

Hereinafter, an aspect in which the hydrogen chloride separation step is performed between the reaction step and the raw material separation step will be described. In a case in which the hydrogen chloride separation step is included, the amount of hydrogen chloride separated in the raw material separation step described above is much smaller than the amount of hydrogen chloride separated in the hydrogen chloride separation step.

The first gas composition may be directly supplied to the hydrogen chloride separation step, or an additional treatment step may be provided between the reaction step and the hydrogen chloride separation step, and the first gas composition subjected to the additional treatment may be supplied to the hydrogen chloride separation step. The additional treatment is a treatment other than separation of hydrogen chloride and water vapor, and a treatment that does not change the composition of substances other than moisture contained in the first gas composition. Examples of the additional treatment include treatments such as storage in a tank, compression by a compressor, heating, and cooling.

Examples of the method of separating hydrogen chloride from the first gas composition include methods such as distillation, adsorption, and neutralization.

Distillation is a method of distilling the first gas composition to separate hydrogen chloride. Distillation can be performed under normal pressure, under increased pressure, or under reduced pressure, but is preferably performed under increased pressure from the viewpoint of improving separation efficiency.

Adsorption is a method of bringing the first gas composition into contact with an adsorbent and adsorbing hydrogen chloride to the adsorbent to separate hydrogen chloride. The adsorbent may be in a solid phase state or in a state (liquid phase) of being dispersed in a liquid medium in which the adsorbent is not dissolved.

Neutralization is a method of bringing the first gas composition into contact with a basic compound and separating hydrogen chloride by causing hydrogen chloride to react. The basic compound may form a solid phase, a liquid phase, or a gas phase, or may be dispersed in a liquid medium. Examples of the basic compound include sodium hydroxide, potassium hydroxide, potassium hydrogen carbonate, potassium carbonate, and ammonia. Sodium hydroxide is preferable from the viewpoint of production cost.

In the hydrogen chloride separation step of subjecting hydrogen chloride to a separation treatment, water vapor may be removed at the same time.

By the hydrogen chloride separation step of subjecting hydrogen chloride to a separation treatment, a gas composition having a lower content ratio of hydrogen chloride than that of the first gas composition is obtained. That is, by the hydrogen chloride separation step, a gas composition having a low content ratio of hydrogen chloride and containing CTFE, HFO-1123, a diluent, and unreacted CFC-113 is obtained.

In a case in which the production method of the present embodiment includes the hydrogen chloride separation step, the gas composition can be applied as the first gas composition. In the gas composition obtained in the hydrogen chloride separation step, in addition to the content ratio of acidic components such as hydrogen chloride and acid fluoride, the content ratio of other components obtained in the reaction step and compounds other than acidic components contained in other compounds produced in the reaction step may be lower than that of the first gas composition.

The gas composition obtained in the hydrogen chloride separation step may be directly supplied to the raw material separation step, or an additional treatment step may be provided between the hydrogen chloride separation step and the raw material separation step, and the gas composition subjected to the additional treatment may be supplied to the raw material separation step. The additional treatment is a treatment other than separation of water vapor, and a treatment that does not change the composition of substances other than moisture contained in the gas composition. Examples of the additional treatment include treatments such as storage in a tank, compression by a compressor, heating, and cooling.

### <Reaction Device>

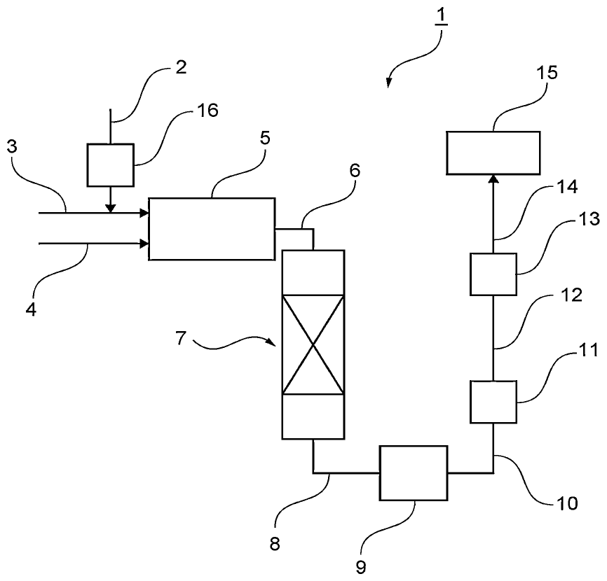

A schematic view of an example of a reaction device used in the production method of the present embodiment is illustrated in FIG. 1.

A reaction device 1 illustrated in FIG. 1 includes a reactor 7 including a heating means, such as an electric heater, for performing a reaction step, and a high-boiling receiver tank 9 for performing the raw material separation step. The reaction device 1 includes, on the downstream side of the high-boiling receiver tank 9, a hydrogen chloride trap 11 for separating hydrogen chloride in the gas composition in the hydrogen chloride separation step, a dehydration device 13 for removing moisture in the gas composition, and a gas collection container 15 for capturing the gas composition.

An upstream side which is an inlet side of the reactor 7 is connected to a preheating mixer 5 including a heating means such as an electric heater. A raw material supply line 6 for supplying a raw material gas from the preheating mixer 5 to the reactor 7 is preferably as short as possible in order to suppress the influence of heat dissipation, and may be kept warm by a heat insulating material.

A supply line 4 for supplying CFC-113 and a supply line 3 for supplying hydrogen are connected to the preheating mixer 5. The diluent is supplied by a diluent line 2, heated by a preheater 16 as necessary, and then supplied to the supply line 3 to be mixed with hydrogen. In particular, in a case in which water vapor is used as a diluent, the water vapor is heated in advance by the preheater 16, vaporized, and supplied to the supply line 3.

A mixed gas 1 of a diluent and hydrogen is supplied to the preheating mixer 5 by the supply line 3. CFC-113 is supplied to the preheating mixer 5 by the supply line 4, heated to a predetermined temperature, vaporized, and mixed with a mixed gas 1 to become a raw material gas.

Examples of the temperature at which CFC-113 is heated to vaporize in preheating mixer 5 include, for example, from 60° C. to 100° C. Examples of the temperature at which the mixed gas 1 is heated in the preheating mixer 5 includes, for example, from 100° C. to 600° C. The raw material gas obtained by mixing CFC-113 and the mixed gas 1 is supplied to the reactor 7 by the raw material supply line 6. CFC-113 in the raw material gas supplied to the reactor 7 is brought into contact with hydrogen in the raw material gas to be converted into CTFE and HFO-1123. In the reactor 7, the first gas composition containing a diluent, hydrogen chloride, unreacted CFC-113 and the like in addition to CTFE and HFO-1123 is obtained.

The first gas composition obtained in the reactor 7 is supplied to the high-boiling receiver tank 9 by a reactor outlet line 8, and a high-boiling component, mainly unreacted CFC-113, is liquefied in the high-boiling receiver tank 9, while the water vapor is also liquefied if used as a diluent. As a result, the second gas composition containing CTFE and HFO-1123 which are low-boiling components is obtained. The liquefaction of CFC-113 and the like in the high-boiling receiver tank 9 is performed by cooling the tank using, for example, a refrigerant. In a case in which water vapor is used as a diluent, examples of the temperature at which the tank is cooled in order to liquefy the water vapor include, for example, 5° C. or lower.

The outlet of the high-boiling receiver tank 9 is connected to the hydrogen chloride trap 11 in which an aqueous alkali solution is housed, by an outlet line 10. The second gas composition obtained in the high-boiling receiver tank 9 is supplied to the hydrogen chloride trap 11, and passes through the hydrogen chloride trap 11 in which an aqueous alkali solution is housed, whereby hydrogen chloride contained in the second gas composition is neutralized by alkali. As a result, the second gas composition from which hydrogen chloride has been removed is obtained. Examples of the alkali include an aqueous sodium hydroxide solution.

The outlet of the hydrogen chloride trap 11 is connected to the dehydration device 13 by an outlet line 12. The second gas composition obtained in the hydrogen chloride trap 11 is supplied to the dehydration device 13. In the dehydration device 13, moisture remaining in the second gas composition is removed by the water trap, and the second gas composition is dried. Examples of the water trap include a porous adsorbent such as a molecular sieve. The second gas composition from which moisture has been removed by the dehydration device 13 is recovered by the gas collection container 15 through an outlet line 14, and then the components contained in the second gas composition are analyzed by an analyzer such as gas chromatography (GC).

HFO-1123 obtained as described above is useful as a refrigerant in place of HFC-32 (difluoromethane) and HFC-125 (pentafluoroethane) which are greenhouse gases.

CTFE obtained as described above is useful as a raw material for HFO-1123, and also for a polymer.

By using water vapor as a diluent, even CTFE and HFO-1123 having a low boiling point (standard boiling point) can be easily separated. As a result, the manufacturing cost is suppressed, and the productivity of HFO-1123 and CTFE is improved.

### EXAMPLES

Hereinafter, embodiments of the present disclosure will be described in detail with reference to Examples; however, the embodiments of the present disclosure are not limited thereto.

### <Reaction Device>

In Examples and Comparative Examples, the same reaction device (hereinafter, referred to as a reaction device (1)) as illustrated in FIG. 1 was used.

### (Reaction Device (1))

In the reaction device (1), a cylindrical vertical reactor made of SUS304 or Inconel 600 (JIS standard) and having an inner diameter of 23.4 mm×a length of 400 mm or an inner diameter of 10.7 mm×a length of 400 mm was used as the reactor 7. Specifically, the reactor 7 made of SUS304 was used in examples in which water vapor was not used, and the reactor 7 made of Inconel 600 was used in examples in which water vapor was used. Only in Example 3, the size of the reactor 7 was set to an inner diameter of 23.4 mm×a length of 400 mm. The inside of the reactor 7 was heated by an electric furnace.

In the raw material supply line 6 connected to the inlet side of the reactor 7, in order to reduce the influence of heat dissipation, the line was made as short as possible and kept warm with a heat insulating material made of ceramic fiber. CFC-113 as a raw material was heated and vaporized at from 60° C. to 100° C. in the preheating mixer 5. The hydrogen gas was mixed with the diluent and then heated at from 100 to 600° C. in the preheating mixer 5 to be mixed with the vaporized CFC-113.

CFC-113 as a raw material was supplied by adjusting the flow rate with a plunger type pump (manufactured by FUJI PUMP CO., LTD.) or a syringe pump (manufactured by YMC. CO., LTD.) (not illustrated) at the supply line 4. Hydrogen as a raw material was supplied by adjusting the flow rate with a mass flow controller installed in the supply line 3. The supply method of the diluent varies depending on the type of diluent used, that is, whether the diluent is nitrogen or water vapor. In a case in which the diluent was nitrogen, nitrogen was supplied by adjusting the flow rate with a mass flow controller installed in the diluent line 2. In a case in which the diluent was water vapor, the flow rate was adjusted by a liquid mass flow controller installed in the diluent line 2 to supply desalted water, and the desalted water was vaporized by the preheater 16 and then mixed with hydrogen.

The reactor outlet line 8 connected to the outlet side of the reactor 7 was heated by a ribbon heater so as to be in a range of from 80 to 1120° C. and connected to the high-boiling receiver tank 9. A part of the gas from the reactor outlet line 8 was collected using a sampling bag made of polyvinylidene fluoride (PVdF), and the composition of the first gas composition obtained from the reactor outlet was analyzed.

In the high-boiling receiver tank 9, the tank was cooled using a refrigerant to condense the high-boiling component. The outlet line 10 connected to the outlet side of the high-boiling receiver tank 9 was connected to the hydrogen chloride trap 11 in which a 20% by mass aqueous sodium hydroxide solution is housed. The outlet line 12 connected to the outlet side of the hydrogen chloride trap 11 was connected to the dehydration device 13, which was filled with 220 g of pellet-like molecular sieves 4A (manufactured by Tosoh Corporation, columnar product 1.5 mm). The outlet line 14 connected to the outlet side of the dehydration device 13 was connected to the gas collection container 15, and the obtained second gas composition was recovered.

### <Analysis Conditions>

Gas chromatography (GC-7890A, manufactured by Agilent Technologies, Inc.) was used for the analysis of the first gas composition obtained from the reactor outlet. DB-1301 (manufactured by Agilent Technologies, a length of 60 m× an inner diameter of 250 μm×a thickness of 1 μm) was used as a column. As a detector, a flame ionization detector (FID) was used. Hereinafter, gas chromatography is also referred to as “GC”.

### <Time of Reaction>

The time of reaction means a time during which CFC-113 and hydrogen as reaction gases remain within a reaction temperature zone in a reactor, and was calculated by dividing a gas flow rate (volume flow rate) by a reactor volume, that is, a cross-sectional area of the reactor and a reaction length. The reaction length was confirmed to be about 200 mm regardless of the reaction conditions by measuring the temperature in the reactor using a thermocouple (manufactured by SAKAGUCHI ELECTRIC HEATERS CO., LTD.).

Time (/s) for reaction=flow rate (mm³/s)/cross-sectional area (mm²)/reaction length (mm)

### Example 1

The temperature in the reactor 7 of the reaction device (1) was set to 575° C., and a mixed gas obtained by mixing 12 vol % of CFC-113, 18 vol % of hydrogen gas, and 70 vol % of water vapor as a diluent was supplied to the reactor 7. The mixed gas was continuously flowed and analyzed in 1, 2, and 3 hours, and it was confirmed that the composition of the first gas composition as an outlet gas was stabilized in 1 hour from the start of the reaction since the analysis result was not changed.

Based on the molar ratio (mol %) of each component in the first gas composition obtained by the analysis by GC, the conversion rate of CFC-113, the selectivity of CTFE, and the selectivity of HFO-1123 were respectively determined as follows.

In the following calculation formula, (CFC-113) in, (CFC-113) out, (CTFE) out, (HFO-1123) out, and (total) out represent molar ratios calculated from the GC area ratios of CFC-113 in the raw material gas, CFC-113 in the first gas composition obtained from the reactor outlet, CTFE in the first gas composition, HFO-1123 in the first gas composition, and the entire organic substances in the first gas composition, respectively. In the present example, the calculation is performed assuming that (CFC-113)in=(total)out.

The molar ratio of each component in the first gas composition was calculated by multiplying the area ratio of each component identified by GC by a detection sensitivity factor measured using a standard substance whose composition ratio is known. The volume ratio between CFC-113 and the diluent in the raw material gas was calculated from the flow rate ratio between CFC-113 and the diluent.

### [Conversion Rate (mol %) of CFC-113]

The conversion rate of CFC-113 refers to a ratio at which CFC-113 is converted into other components, including CTFE and HFO-1123, by the reaction and thereby consumed. The conversion rate of CFC-113 is calculated by the following equation.

$$Conversion rate ( mol \% ) of CFC - 113 = \{ 1 - ( CFC - 113 ) out / ( CFC - 113 ) in \} \times 100$$

### [Selectivity (mol %) of CTFE]

The selectivity of CTFE refers to a ratio at which the reacted CFC-113 is converted into CTFE. The selectivity of CTFE is calculated by the following equation.

$$Selectivity ( mol \% ) of CTFE = ( CTFE ) out / \{ 1 - ( CFC - 113 ) out / ( CFC - 113 ) in \} \times 100$$

### [Selectivity (mol %) of HFO-1123]

The selectivity of HFO-1123 refers to a ratio at which the reacted CFC-113 is converted into HFO-1123. The selectivity of HFO-1123 is calculated by the following equation.

$$Selectivity ( mol \% ) of HFO - 1123 = ( HFO - 1123 ) out / \{ 1 - ( CFC - 113 ) out / ( CFC - 113 ) in \} \times 100$$

These results were taken as average values of analysis results of samples collected during the period from the stabilization of the reaction to the end of the reaction.

The calculation results of the conversion rate of CFC-113, the selectivity of CTFE, the selectivity of HFO-1123, and the total selectivity of (CTFE+HFO-1123) are shown in Table 1 together with the reaction conditions, that is, the flow rate proportion (vol %) of CFC-113 supplied to the reactor, the flow rate proportion (vol %) of hydrogen, the type of the diluent, the flow rate proportion (vol %) of the diluent, the temperature (° C.) of the reaction, and the time of the reaction.

The temperature of the reaction is an internal temperature of the reactor 7, and is a measured value by a thermocouple. The time of the reaction is a time during which CFC-113 as a raw material and hydrogen are brought into contact with each other in a reaction temperature zone, and is a value determined by the above-described method.

### Examples 2 to 13

The reaction was continuously performed in the same manner as in Example 1 except that the reaction conditions were changed as shown in Table 1. The conversion rate of CFC-113, the selectivity of CTFE, the selectivity of HFO-1123, and the total selectivity of (CTFE+HFO-1123) were respectively determined in the same manner as in Example 1. The obtained results are shown in Table 1.

In Table 1, “-” indicates that the diluent was not used.

TABLE 1

| | | | | | | | Reaction result | | | |
|---|---|---|---|---|---|---|---|---|---|---|
| | | | | | | | CFC-113 | | | |
| | Temperature | Raw material flow rate proportion | | | | Time for | conversion | CTFE | HFO-1123 | Total |
| | of reaction | CFC-113 | Hydrogen | Diluent | | reaction | rate | selectivity | selectivity | selectivity |
| | (° C.) | (vol %) | (vol %) | (vol %) | Diluent type | (sec) | (mol %) | (mol %) | (mol %) | (mol %) |
| Example 1 | 575 | 12 | 18 | 70 | Water vapor | 6 | 75.5 | 84.2 | 9.4 | 93.6 |
| Example 2 | 575 | 22 | 34 | 44 | Water vapor | 6 | 83.6 | 46.5 | 27.0 | 73.5 |
| Example 3 | 575 | 12 | 18 | 70 | Nitrogen | 6 | 87.5 | 59.2 | 18.0 | 77.2 |
| Example 4 | 575 | 22 | 34 | 44 | Nitrogen | 6 | 77.0 | 45.0 | 28.2 | 73.2 |
| Example 5 | 575 | 40 | 60 | 0 | — | 8 | 74.4 | 37.1 | 19.0 | 56.1 |
| Example 6 | 575 | 40 | 60 | 0 | — | 1 | 31.0 | 51.8 | 23.4 | 75.2 |
| Example 7 | 575 | 75 | 25 | 0 | — | 1 | 19.4 | 68.5 | 13.7 | 82.2 |
| Example 8 | 575 | 59 | 41 | 0 | — | 1 | 30.6 | 56.8 | 19.7 | 76.5 |
| Example 9 | 575 | 50 | 50 | 0 | — | 1 | 30.9 | 54.1 | 23.1 | 77.2 |
| Example 10 | 575 | 40 | 60 | 0 | — | 1 | 31.0 | 51.7 | 23.4 | 75.1 |
| Example 11 | 575 | 33 | 67 | 0 | — | 1 | 30.5 | 52.6 | 23.5 | 76.1 |
| Example 12 | 560 | 40 | 60 | 0 | — | 2 | 22.6 | 59.3 | 20.1 | 79.4 |
| Example 13 | 585 | 40 | 60 | 0 | — | 2 | 47.8 | 46.5 | 24.1 | 70.6 |

In the above examples, Examples 1 to 4 are Examples according to the present disclosure, and Examples 5 to 13 are Comparative Examples. As shown in Table 1, in Examples 1 to 4, as compared with Examples 5 to 13, both improvement in a conversion rate of CFC-113 and improvement in a total selectivity of CTFE and HFO-1123 are achieved.

The entire contents of the disclosures by Japanese Patent Application No. 2022-151981 filed on Sep. 22, 2022 are incorporated herein by reference. All the literature, patent applications, and technical standards cited herein are also herein incorporated to the same extent as provided for specifically and severally with respect to an individual literature, patent application, and technical standard to the effect that the same should be so incorporated by reference.

### DESCRIPTION OF REFERENCE NUMERALS

1 Reaction device
2 Diluent line
3, 4 Supply line
5 Preheating mixer
6 Raw material supply line
7 Reactor
8 Reactor outlet line
9 High-boiling receiver tank
10, 12, 14 Outlet line
11 Hydrogen chloride trap
13 Dehydration device
15 Gas collection container
16 Preheater

1. A production method for producing chlorotrifluoroethylene and trifluoroethylene, the method comprising reacting 1,1,2-trichloro-1,2,2-trifluoroethane with hydrogen in a gas phase in the presence of a diluent,
wherein a volume of the diluent supplied to a reactor at which the reaction is performed is from 0.4 times to 6 times a volume of the 1,1,2-trichloro-1,2,2-trifluoroethane supplied to the reactor.

2. The production method according to claim 1, wherein the diluent includes at least one selected from the group consisting of water vapor, nitrogen, argon, and helium.

3. The production method according to claim 1, wherein the diluent includes water vapor.

4. The production method according to claim 1, wherein a catalyst is not provided inside the reactor.

5. The production method according to claim 1, wherein the volume of the diluent supplied to the reactor is from 3 times to 6 times the volume of the 1,1,2-trichloro-1,2,2-trifluoroethane supplied to the reactor.

6. The production method according to claim 1, wherein a volume of the hydrogen supplied to the reactor is from 0.3 times to 2 times the volume of the 1,1,2-trichloro-1,2,2-trifluoroethane supplied to the reactor.

7. The production method according to claim 1, wherein a temperature of the reaction is from 560° C. to 600° C.

8. The production method according to claim 1, wherein a time of the reaction is from 1 second to 12 seconds.

9. The production method according to claim 3, comprising removing at least a part of the water vapor from a gas composition obtained by the reaction, the gas composition comprising the chlorotrifluoroethylene, the trifluoroethylene, and the water vapor.

10. The production method according to claim 9, wherein the removing includes liquefying at least a part of the water vapor at a temperature of 5° C. or lower.

11. The production method according to claim 1, wherein the 1,1,2-trichloro-1,2,2-trifluoroethane is obtained by fluorinating tetrachloroethylene with hydrogen fluoride in the presence of a catalyst.

12. The production method according to claim 3, wherein a catalyst is not provided inside the reactor.

13. The production method according to claim 3, wherein the volume of the diluent supplied to the reactor is from 3 times to 6 times the volume of the 1,1,2-trichloro-1,2,2-trifluoroethane supplied to the reactor.

14. The production method according to claim 3, wherein a volume of the hydrogen supplied to the reactor is from 0.3 times to 2 times the volume of the 1,1,2-trichloro-1,2,2-trifluoroethane supplied to the reactor.

15. The production method according to claim 3, wherein the 1,1,2-trichloro-1,2,2-trifluoroethane is obtained by fluorinating tetrachloroethylene with hydrogen fluoride in the presence of a catalyst.

16. The production method according to claim 5, wherein a volume of the hydrogen supplied to the reactor is from 0.3 times to 2 times the volume of the 1,1,2-trichloro-1,2,2-trifluoroethane supplied to the reactor.
